(19) United States
(12) Patent Application Publication
(10) Pub. No.: US 2021/0046250 A1
(43) Pub. Date: Feb. 18, 2021

(54) Method for Monitoring a Quantity of Medication Liquid in a Syringe, a Syringe Using the Method, and a Plunger Part for Use in the Syringe

(71) Applicant: Missio B.V., Valkenswaard (NL)

(72) Inventor: Josephus Joannes Antoinius Geboers, Valkenswaard (NL)

(21) Appl. No.: 16/966,920

(22) PCT Filed: Jan. 30, 2019
(86) PCT No.: PCT/EP2019/052273

§ 371 Date: Aug. 3, 2020

(30) Foreign Application Priority Data
Feb. 1, 2018 (EP) 18154589.8

(51) Int. Cl. A61M 5/315

(52) U.S. Cl. CPC A61M 5/31568; A61M 2205/52; A61M 2205/8206; A61M 2205/3331; A61M 2205/332; A61M 2205/3584

(57) ABSTRACT

A method is provided for monitoring a quantity of a medication liquid stored in a syringe. The syringe includes a tube shaped syringe body with an open end and distal end opposite to the open end and configured to store the medication liquid, and a plunger positioned in the syringe body. The plunger forms a seal with an inner wall of the syringe body and is causes a discharge of the medication liquid though a discharge opening of the syringe body when a force is exerted on the plunger. In the method a parameter is derived which is indicative of the pressure exerted to the liquid. Quantity information indicative of a quantity of medication liquid discharged is derived from the derived parameter and the time duration that the pressure indicated by the parameter is exerted.

### FIELD OF THE INVENTION

The invention relates to a method for monitoring a quantity of medication liquid in a syringe, a syringe using the method, and a plunger part for use in the syringe.

### BACKGROUND

The present invention relates to the field of medication delivery, and more particularly, to medication delivery by means of a syringe.

Patients who must inject with a medicinal product can face different problems. The failure to carefully fulfil the prescribed therapy causes very high cost in the national healthcare. Failure to follow a therapy may have different causes such as avoiding side effects or simply forgetting the medication. Sometimes certain therapies are complex in terms of use, the time and date when the product is to be used and when it has to be stopped. When patients would have a simple and reliable tool, that could save a lot of costs and above all, a lot of suffering. The use of sometimes very expensive medicines and the efficacy of the drug can be very much improved, even more when using electronic tools. These tools should be very friendly in use, and work extremely reliably.

### SUMMARY OF INVENTION

It is an object of the invention to provide a simple and reliable method and tool which helps patient to monitor a correct administration of a medication when a syringe is used.

According to a first aspect of the invention the object is achieved by a method for monitoring a quantity of a medication liquid stored in a syringe, comprising a tube shaped syringe body with an open end and distal end opposite to the open end and configured to store the medication liquid, and a plunger positioned in the syringe body and forming a seal with an inner wall of the syringe body, the plunger being movable to cause a discharge of the medication liquid though a discharge opening of the syringe body when a force is exerted on the plunger, in the method a parameter is derived which is indicative for the pressure exerted to the liquid, further quantity information indicative for a quantity of medication liquid discharged is derived from the derived parameter and the time duration that the pressure indicated by the parameter is exerted.

According to a second aspect of the invention this object is achieved by a syringe, comprising a tube shaped syringe body with an open end and distal end opposite to the open end and configured to store a medication liquid, and a plunger positioned in the syringe body and forming a seal with an inner wall of the syringe body, the plunger being movable to cause a discharge of the medication liquid though a discharge opening in the distal end of the syringe body, the syringe further comprising a subsystem for deriving a parameter which is indicative for a pressure exerted on the medication liquid and electronic circuitry for deriving quantity information indicative for a quantity of medication liquid discharged on the basis of the parameter and time duration that the pressure indicated by said parameter is exerted on the medication liquid.

During the production of the syringe, small deviations may occur in the dimensions of the individual parts. These deviations may result in deviations of friction between the syringe body and the plunger body during use. Such deviations in the friction may result in less accurate indications of quantities based on the measured pressure over time. To improve the indication of discharged liquid quantity, the system may be configured to measure an initial indication for friction, during the filling of the liquid into the syringe. This indication for friction between the plunger and the syringe body may subsequently be stored into a memory in the system. This indication may be the indication of the specific individual syringe, but may also be an average indication over a batch of syringes of which the individual syringe is part of. Such indication of friction during filling of the syringe, i.e. during the backward movement of the plunger to take in liquid into the syringe body, may be used to calibrate and/or correct for other indications of that syringe, such as e.g. the indication of discharged liquid based on exerted pressure during use by an end-user.

In a further embodiment, the plunger may comprise means for determining an end-position of the plunger in the syringe body. This improves the accuracy of the determination of the quantity of discharged liquid. Such means for determining an end-position or end-region, may e.g. comprise a capacitive measurement such as measuring one or more metal objects such as rings mounted on the outside of the syringe by a sensor inside the plunger. This sensor may be mounted on a predetermined position anywhere in the plunger such as at or in the thumb knob, on the opposite end of the plunger or anywhere in between. The addition of such a target position or region detection, enriches the information that can be read from the syringe memory in order to determine the specific quantity and/or specific date and time of the discharge.

According to a third aspect of the invention the object is achieved by a plunger part for use in combination with a tube shaped syringe body for storing a medication liquid, comprising a plunger to be moveably positioned in the syringe body and thereby forming a seal with an inner wall of the syringe body, the plunger comprising a subsystem for deriving a parameter which is indicative for a pressure exerted on the medication liquid and electronic circuitry for deriving quantity information indicative for a quantity of medication liquid discharged on the basis of the parameter and time duration that the pressure indicated by said parameter is exerted on the medication liquid.

The method and syringe according to the invention provides electronically readable information about the quantity of medication liquid in the syringe which is helpful to detect correct or incorrect usage of the syringe. The method enables the possibility to remain the current used materials for syringes which are in direct contact with the medication.

In an embodiment of the method a memory is used for storing the quantity information.

This embodiment has the advantage that the actual quantity of medication is always accessible in an electronic way, which enables checking afterwards whether all medication liquid has been administered.

In a further embodiment of the method together with the quantity information, time information indicating the moment at which a quantity indicated by said quantity information has been reached, is stored in the memory.

This embodiment has the advantage that afterwards it can checked at which moment the administration of the medication liquid has been done. This is very helpful for monitoring the therapy compliance.

In an embodiment of the method a wireless interface is used between the syringe and a device with a user interface for providing the information stored in the memory to a user.

This embodiment has the advantage that the information in the memory can easily be read out by an external device. In particular a smart phone is very suitable since such phones are in general available for the patients and apps for reading out the memory and rendering the information in visible or audible manner can be easily developed and installed on a smart phone.

### BRIEF DESCRIPTION OF THE DRAWINGS

These and other aspects of the invention will be apparent from and elucidated further with reference to the embodiments described by way of example in the following description and with reference to the accompanying drawings, in which

FIG. 1 shows an embodiment of a syringe,

FIG. 2 shows an embodiment of a plunger part for use in the syringe according to the invention,

FIG. 3 shows an electronic diagram of an embodiment of electronic circuitry for use in the syringe according to the invention,

FIG. 4 shows a flow diagram of program performed by an embodiment of a program controlled controller for use in the syringe according to the invention,

FIG. 5a shows an example of samples of pressure exerted on a medication liquid during the disposal of the medication liquid,

FIG. 5b, shows the quantity of the medication liquid in the syringe calculated by the electronic circuitry on the basis of the pressure samples, and FIG. 6 shows an energy saving embodiment of electronic circuitry for use in the syringe according to the invention.

The figures are schematic in nature and are not drawn to scale. In particular, for the sake of clarity, some dimensions are exaggerated to a greater or lesser extent. Corresponding parts are designated in the figures as far as possible designated by the same reference numeral.

### DETAILED DESCRIPTION OF EMBODIMENTS

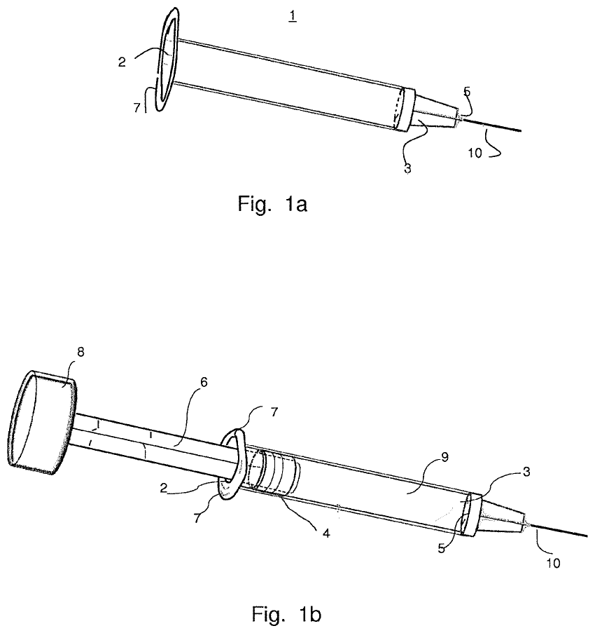

FIG. 1a shows a tube shaped syringe body 1 with an open end 2 and a distal end 3 with an opening 5 to which an injection needle 10 is connected. FIG. 1b shows a syringe comprising the syringe body 1 and a plunger 4 positioned in the syringe body 1. The plunger 4 forms a seal with the inner wall of the syringe body 1 and is movable in the direction of the distal end 3. A medication liquid 9 is stored in the syringe body 1 in the space between the distal end 3 of the tube shaped syringe body 1 and the plunger 4. One end of an injector piston 6 is engaged with the plunger 4, whilst the other end of the injector piston 6 is provided with a thumb knob 8. The syringe body 1 is provided with finger grips 7 which together with the thumb knob 8 enables to exert a force on the plunger 4 with one hand, resulting in a movement of the plunger 4 in the direction of the distal 3 and consequently a disposal of the medication liquid 9 via the opening 5 and injection needle 10.

There is a need to automatic monitoring the quantity of medication liquid still present in the syringe. For such automatic monitoring a monitor signal that can be processed by electronic signal processors is needed. According to the invention this can be provided in an reliable and inexpensive manner on the basis of the pressure exerted on the medication liquid 9 during the disposal and the time that this pressure is exerted. The speed with which the medication liquid is disposed is determined by the pressure exerted. So the mathematical time integral of this speed is indicative for the amount of medication liquid 9 which is already disposed. The quantity of the medication liquid in a full syringe, hereinafter referred as starting quantity Qₛ, is known. So the actual quantity of medication liquid still present in the syringe can be easily determined by subtracting the disposed quantity from the starting quantity Qₛ. Quantity information, indicating the actual quantity of medication liquid in the syringe is stored in an electronic memory.

The pressure can be measured in different ways, from the direct force on the plunger exerted by the person to put the plunger in motion or by the counter-pressure exerting the liquid on the plunger when it is put into motion. Both methods will give a similar result.

Note that the invention is not limited to pressure measurements solutions mentioned above. Other solutions for determining the pressure can be used. For example by means of a pressure sensor placed on the inner side of the syringe body. It is also possible to measure the force exerted on the piston. This force divided by the size of the area of the plunger which is in contact with the medication liquid gives a measure for the pressure. However this is less accurate than directly measuring the pressure because of friction forces between the plunger and syringe body. A location of the pressure sensor in the plunger is preferred because it is a simple solution which results in a reliable and accurate measuring of the pressure.

The monitor signal is very suitable to determine the time and date when the modification has been completely administered. This can be done by determining whether the quantity information represents a level which is below a minimum quantity Qₘᵢₙ for which the syringe can be regarded as empty and storing in a memory date information representing the moment in time that this minimum level has been reached. Later on the stored date information can read from the memory to check the date and time of medication administration.

Preferably the syringe is provided with the memory and a wireless interface making the information stored in the memory accessible for an external device. A smart phone with a wireless interface can be used which is provided with an app for reading out the information stored in the memory and displaying on its screen information indicating how much of the medication is already disposed or still present in the syringe together with the date and time that this has been reached. In order to enable the calculation of the amount of quantity still present in the syringe the value of the start quantity Qₛ is stored in the memory. The quantity Q can be calculated by subtracting each time that a sample is taken a reduction value dQ caused by the sampled pressure during an time interval equal to the distance between two samples. For checking the therapy compliance in many situations it is sufficient to know when medication liquid has been administered and whether all medication liquid has been administered. In those situations it suffices to store an indication that the syringe can be regarded as empty together with the date that this empty level has been reached.

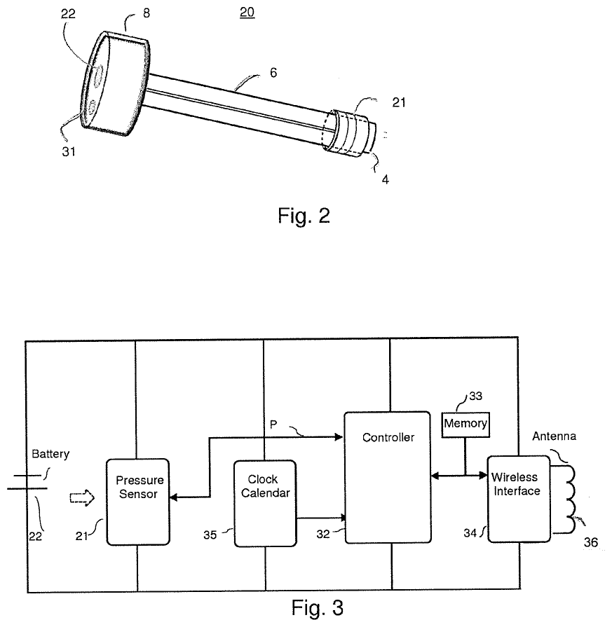

FIG. 2 shows a plunger part 20 of a syringe according to the invention for use in combination with the tube shaped syringe body 1. The plunger 4 is provided with a pressure sensor 21 for measuring the pressure P exerted on the medication liquid during the disposal of the medication liquid 9. The construction of the plunger 4 can be adjusted so that the sensor is inserted into the plunger material so that the sensor is protected from external influences. The syringe is further provided with an electronic circuit 31 for deriving the quantity information on the basis of the pressure P and the time that this pressure is exerted. Preferably this electronic circuitry 31 is located in the thumb knob 8. The thumb knob 8 is further provided with a battery compartment for storing a battery 22 for energizing the electronic circuit 31. The location of the electronic circuitry 31 and the battery compartment is preferably in the thumb knob 8, but it will be clear for the skilled person that those part can be located at other locations in the syringe.

FIG. 3 shows an electronic diagram of an suitable embodiment of the electronic circuitry 31. The circuitry 31 comprises a clock/calendar circuit 35, a controller 32, a memory 33 and a wireless interface circuit 34. The clock/calendar circuit 35 is of a usual type that generates a signal indicating the actual date and time. The controller 32 is of a program controlled type loaded with a program for determining the quantity information Q on the basis of the pressure P determined by a subsystem comprising a pressure sensor 21 for deriving a parameter which is indicative for the sensed pressure. The controller 32 determines the quantity information Q on the basis of the sensed pressure and the time duration that the pressure is exerted and stores the quantity information Q in the memory 33 together with date information indicating the corresponding actual date. Wireless interface circuit 34 is of a usual type which is able to communicate with an wireless interface of an external device for transferring via an antenna information stored in the memory 33 to the external device. A very suitable location to place the antenna 34 is the piston 6 of the syringe. The controller 32 is configured to sample the pressure P sensed by pressure sensor 21 at equidistant sample moments tn.

FIG. 4 shows a flow diagram of an embodiment of a controller program for determining and storing the quantity information Q. In step S1 a sample Pₙ of the pressure P is taken. In step S2 the reduction value dQ caused by the pressure Pₙ in the time interval dt between two subsequent samples is calculated. Although the relation between the pressure P and the quantity reduction value dQ. is not linear, in most practical situations the relation can be approximately represented by linear one. dQ is calculated using the equation 1.

dQ=K×P (equation 1),

with K a constant determined by the outflow resistance of the syringe. This resistance is determined by the physical dimensions of the syringe body 1, opening 5 and needle 10 and further the viscosity of the medication liquid.

As mentioned hereinbefore this resistance is not exactly linear with the pressure P. For a more accurate calculation of the update of the quantity a look-up table with the exact relation between the pressure P and the quantity reduction dQ can be used.

In step S3 the value of Q in memory 33 is updated according to equation 2 and stored in the memory 33.

Qₙ=Qₙ₋₁−dQₙ (equation 2)

In step S4 the actual date and time at the output the clock/calendar circuit 35 is read by the controller 32 and stored as date information in the memory 33.

In step S5 it is checked whether the updated quantity is below a minimum value Qₘᵢₙ, which indicates that the syringe is considered to be empty. If not then in step S6 it is checked whether an “empty”-flag, indicating that the syringe is empty, in memory 33 has been set. If not then program proceed with step S1 in which a subsequent sample Pₙ of the pressure P is taken on the next sample moment tₙ.

The loop of S1 to S6 is continued until in S5 it is established that Q is below Qₘᵢₙ, which means that the syringe is to be considered as empty. Then step S6 is followed by step S7 in which the “empty”-flag is set in the memory 33. Step S7 is followed by step S6 in which now it is established that the “empty”-flag is set and subsequently the program is stopped in step S8.

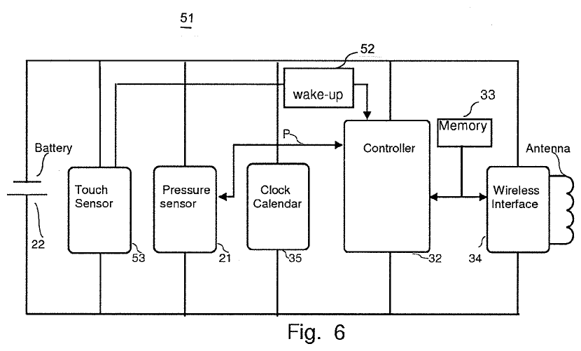

FIG. 5a shows with curve 40 an example of the pressure P as function of the time t during the disposal of the medication liquid when a force is exerted on the plunger 4, starting at moment tₛ. The controller 32 takes samples at equidistant sample moments to (n=1, . . . , 15). The samples with sample values Pₙ are indicated by reference sign 41. After each sample the quantity Q is decreased with the value dQₙ, derived from the corresponding sample value Pₙ. The value of the quantity Q is shown in FIG. 5b as function of the time t. As soon as the value of Qn becomes below Qₘᵢₙ the “empty”-flag is set. This point is indicated in FIG. 5b with reference sign 43.

FIG. 6 shows another embodiment of the electronic circuitry 51 for use in the syringe according to the invention. In FIG. 6 the parts which are identical with the embodiment of FIG. 3 are indicated with same reference numbers. The electronic circuitry is extended with a wake-up circuit 52 and a touch sensor 53. The touch sensor is located at the exterior of the syringe at a location which is touched by user when an injection is given. Preferably the touch sensor 53 is located at the surface of the thumb knob 8, but it will be clear for the skilled person that other locations or possible, for example on one of the finger grips.

The touch sensor 53 is of usual type that generates a touch signal as soon as it is touched by the user. The touch signal is submitted to an input of the wake-up circuit 52. The wake-up circuit 52 is of an usual type that can switch the electronic circuitry 51 between a stand-by mode with low power consumption wherein a part of the circuitry for deriving the quantity information (in this embodiment the controller 32) is inactive and an active mode wherein said part is active. The wake-up circuit 52 is configured to set the controller 32 in the active mode in response to the touch signal on its input and to set the controller in the inactive mode in case the touch signal is absent during a certain time interval.

With the use of the wake-up circuit a substantial amount of battery energy is saved during the storage time of the syringe, which can be very long. Storage times of several months are not unusual.

It is beneficial to combine the electronic circuitry 51 or 50 with additional circuitry for recording storage conditions such as temperature of the medication liquid. For measuring the temperature a temperature sensor can be mounted in the plunger preferably as close as possible to the medication fluid, so that the exact temperature of the medication can be recorded and stored on programmable interval times into the electronic memory 33. Also, here it is advisable to incorporate the temperature sensor into the material of the plunger so that the external influences are negligible.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality. A single processor or controller or other unit can fulfil the functions of several items recited in the claims. The mere fact that certain measures are recited in mutually different dependent claims does not indicate that a combination of these measures cannot be used to advantage. Any reference symbol in the claims should not be construed as limiting the scope.

What is claimed is:

1. Method for monitoring a quantity of a medication liquid stored in a syringe of the type comprising a tube shaped syringe body with an open end and a distal end opposite to the open end and configured to store the medication liquid, and a plunger positioned in the syringe body and forming a seal with an inner wall of the syringe body, the plunger being movable to cause a discharge of the medication liquid though a discharge opening of the syringe body when a force is exerted on the plunger, the method comprising the steps of:
deriving a parameter which is indicative of the pressure exerted on the liquid, and
deriving further quantity information indicative of a quantity of medication liquid discharged from the derived parameter and the time duration that the pressure indicated by the parameter is exerted.

2. Method according to claim 1 further comprising the step of storing the quantity information in a memory.

3. Method according to claim 2 further comprising the step of storing, together with the quantity information, time information, indicative for a moment, that a quantity indicated by said quantity information has been reached in the memory.

4. Method according to claim 2, further comprising the step of providing the information stored in the memory to a user, via a wireless interface between the syringe and a device with a user interface.

5. Method according to claim 2, wherein the quantity information stored in the memory indicates that substantially all or part of the medication liquid has been discharged.

6. Method according to claim 1, further comprising the step of determining the pressure on the medication liquid by a pressure sensor placed positioned in the plunger.

7. A syringe comprising:
a tube shaped syringe body with an open end and a distal end opposite to the open end and configured to store a medication liquid,
a plunger positioned in the syringe body and forming a seal with an inner wall of the syringe body, the plunger being movable to cause a discharge of the medication liquid though a discharge opening in the distal end of the syringe body,
a subsystem for deriving a parameter which is indicative of a pressure exerted on the medication liquid and
electronic circuitry for deriving quantity information indicative of a quantity of medication liquid discharged on the basis of the parameter and time duration that the pressure indicated by said parameter is exerted on the medication liquid.

8. A syringe according to claim 7 further comprising a memory, whereby the electronic circuitry is configured to store quantity information in the memory.

9. A syringe according to claim 8
further comprising a clock subsystem for providing date information indicating the actual time and date,
whereby the electronic circuitry is configured to store together with the quantity information, the time and date information provided by the clock system at a moment that a quantity indicated by said quantity information, has been reached.

10. A syringe according to claim 8, wherein the electronic circuitry is configured to store in the memory, information indicating that the syringe is empty in response to a detection that the quantity information indicates that substantially all medication liquid has been discharged.

11. A syringe according to claim 7, wherein the subsystem for deriving the parameter comprises a pressure sensor for measuring the exerted pressure.

12. A syringe according to claim 11, whereby the pressure sensor is positioned in the plunger.

13. A syringe according to claim 7, whereby the subsystem is arranged to derive the parameter from the force exerted on the plunger for discharging the medication liquid, whereby the subsystem includes a force sensor for sensing said force.

14. A syringe according to claim 7, wherein the syringe includes a wireless interface making the information stored in the memory accessible by an external device with a user interface for providing the information to a user.

15. A syringe according to claim 14 further comprising an injector piston configured to engage the plunger as it moves within the syringe body to expel the medication liquid through the opening in the syringe body, which piston includes an antenna of the wireless interface for communicating with the external device.

16. A syringe according to claim 7
wherein the electronic circuitry is switchable between a standby mode with low power consumption wherein a part of the circuitry for deriving the quantity information is inactive and an active mode wherein said part is active,
wherein the syringe further includes a touch sensor generating a touch signal when it is touched by a human being and
wherein the circuitry further includes a wake-up circuit for temporary switching the circuitry in the active mode in response to the touch signal.

17. A syringe according to claim 16,
wherein the syringe further includes a finger grip connected to an exterior of the syringe body and
further comprising a thumb knob connected at one end of the injector piston opposite to the end engaged with the plunger for enabling to discharge the medication liquid with one hand, and wherein the thumb knob is provided with the touch sensor.

18. A syringe according to claim 7,
wherein the syringe includes a finger grip connected to an exterior of the syringe body and
further comprising a thumb knob connected at one end of the injector piston opposite to the end engaged with the plunger for enabling to discharge the medication liquid with one hand,
whereby one of the following is located in the thumb knob:
the electronic circuitry,
a compartment for storing a battery for energizing the electronic circuitry, and
both the electronic circuitry and the compartment.

19. A syringe according to claim 7, whereby the syringe body contains the medication liquid.

20. A plunger part for use in combination with a tube shaped syringe body for storing a medication liquid, comprising:
a plunger adapted to be moveably positioned in the syringe body and thereby forming a seal with an inner wall of the syringe body, the plunger comprising:
a subsystem for deriving a parameter which is indicative of a pressure exerted on the medication liquid and
electronic circuitry for deriving quantity information indicative of a quantity of medication liquid discharged on the basis of the parameter and time duration that the pressure indicated by said parameter is exerted on the medication liquid.

21. A plunger part according to claim 20, whereby the subsystem for deriving the parameter comprises a pressure sensor for measuring the exerted pressure, which pressure sensor is located in the plunger.

22. A plunger part according to claim 20, further comprising an injector piston configured to engage the plunger as it moves within the syringe body to expel the medication liquid through the opening in the syringe body, which piston including a thumb knob connected at one end of the injector piston opposite to the end engaged with the plunger, whereby one of the following is located in the thumb knob:
the electronic circuitry,
a compartment for storing a battery for energizing the electronic circuitry, and
both the electronic circuitry and the compartment.

23. A plunger part according to claim 22, further including a wireless interface making for the information stored in the memory accessible by an external device with a user interface for providing the information to a user, whereby the piston includes an antenna of the wireless interface for communicating with the external device.
